(19) United States
(12) Patent Application Publication
(10) Pub. No.: US 2026/0245712 A1
(43) Pub. Date: Aug. 20, 2026

(54) GROSS DISSECTION BENCH TECHNOLOGY PLATFORM APPLICATION

(71) Applicant: MP Acquisition, LLC, Madison Heights, MI (US)

(72) Inventor: Michael Scott McClain, Waterford, MI (US)

(73) Assignee: MP Acquisition, LLC, Madison Heights, MI (US)

(21) Appl. No.: 19/057,054

(22) Filed: Feb. 19, 2025

(51) Int. Cl. G16H 40/63; A61B 16/00; G16H 10/60

(52) U.S. Cl. CPC G16H 40/63; A61B 16/00; G16H 10/60

(57) ABSTRACT

A grossing station having a built-in operating system to host multiple software and technology applications is disclosed. The grossing station software platform may be for use in integrating workflow technologies into an electronic health record. The grossing station software platform may comprise a host operating system that allows the independent loading, installation and validation of individually technology applications on a single grossing station. This may allow for the software platform to be loaded on a separate computer that is integrated into the grossing station. This may enable the installation and validation of the grossing station to be separately validated for any future medical device technology applications that might be added in the future. This may have the advantage of validating the connection to the electronic health records separately on the workbench platform versus having to validate every individual technology running on the workbench at the time of installation.

### BACKGROUND OF THE INVENTION

### 1. Field of the Invention

The present invention relates generally to pathology workflows, specifically to a software platform used with a gross dissection bench or grossing station.

### 2. Description of Related Art

Pathology workflows, especially in gross dissection, have become increasingly complex with the advent of new imaging and diagnostic technologies. Additionally, these technologies operate in isolation requiring separate validation and integration into the laboratory environment. This lack of seamless integration complicates validation processes, especially when technologies are classified as medical devices and need to comply with specified FDA regulations.

The challenge lays in the fact that the dissection tools and technologies used on the grossing station or gross bench are not well integrated into the overall hospital network system, including electronic health records (EHR) systems. Existing workflows require the individual validation of each tool and technology at the time of installation, which is both time consuming and costly. Therefore, there is a need in the art for a gross dissection bench or grossing station that has a platform that enables integration of multiple technologies and applications within the laboratory process, particularly for use in digital pathology and other imaging technologies and provides the means for separately validating individual applications for connection to the EHR.

### SUMMARY OF THE INVENTION

One object of the present invention may be to provide an improved grossing station.

Another object of the present invention may be to provide a grossing station that has a technology platform application integrated therewith.

Yet a further object of the present invention may be to provide a grossing station that has a common operating system, wherein individual applications that link to new technology may be loaded individually and therefore separately validated into the laboratory process.

Still another object of the present invention may be to provide a grossing station that has a software platform that would likely be loaded on a separate computer or mini computer, which is integrated into the grossing station.

According, to the present invention, the foregoing and other objects and advantages are obtained by a novel apparatus and methodology that provides a grossing station software platform and separate computer or mini computer designed to facilitate the integration of various technologies into the pathology workflow while allowing these technologies to be separately validated. This software platform may run on an embedded computer system or mini computer integrated into the grossing station or gross dissection bench. By serving as a common operating system for the grossing station, the software platform may allow for the loading, installation and validation of individual software applications without the need to validate the entire system upon every new technology being added thereto. Furthermore, the software platform of the grossing station of the present invention may provide flexibility incorporating emerging technology, such as but not limited to, digital cameras for digital pathology workflows, advanced imaging systems like MicroCT, SWIR cameras, ultrasound, x-ray, and others. Each of these technologies may require a specific software application, which may be validated separately for FDA approval, thereby streamlining the integration of future medical technologies while also ensuring compliance with regulatory standards.

One advantage of the present invention may be that it provides for an improved grossing station.

A further advantage of the present invention may be that it provides for an improved grossing station that uses a technology software platform application therewith.

Still another advantage of the present invention may be that it provides for a grossing station that runs on a common operating system, wherein an individual application that is associated with a new technology may be loaded individually and therefore separately validated into the laboratory process.

Still another advantage of the present invention may be that it provides a grossing station that would include a separate computer or mini computer integrated into the grossing station which would enable the installation and validation of applications of the grossing station to be separately validated for any future medical device technology that might be added.

Other objects, features and advantages of the present invention may become apparent from the subsequent description, and the appended claims taken in conjunction with the accompanying drawings.

### BRIEF DESCRIPTION OF THE DRAWINGS

FIG. 1 shows a plan view of a grossing station according to the present invention.

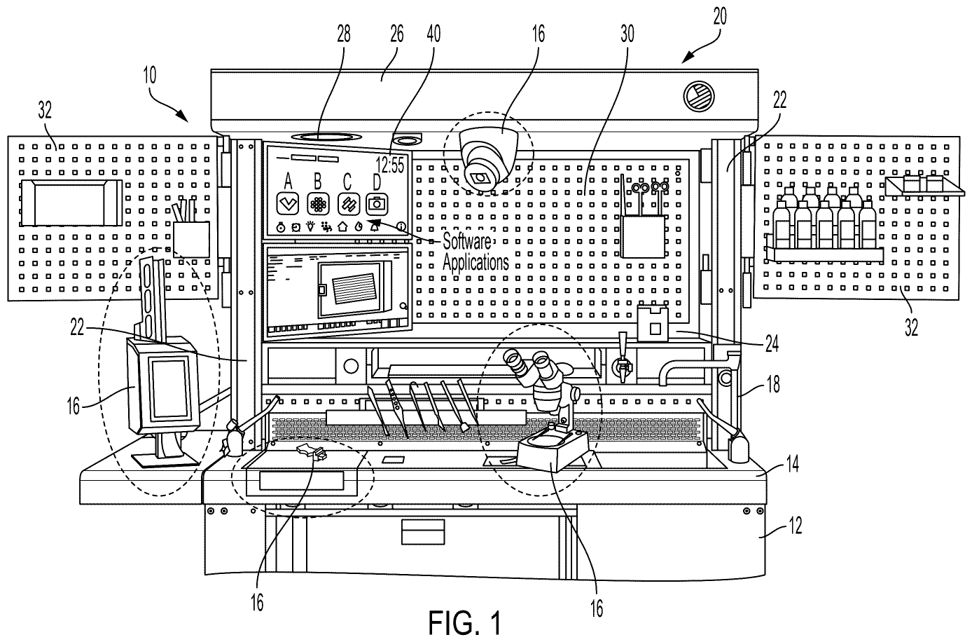

FIG. 2 shows a flowchart of a grossing station platform which hosts validated software applications according to the present invention.

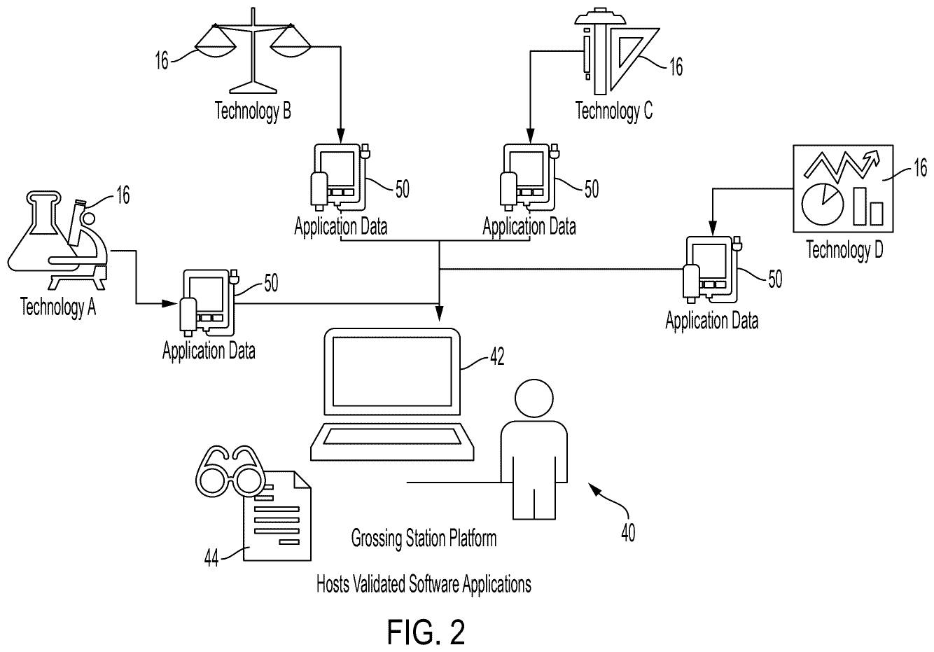

### BACKGROUND OF THE INVENTION

Referring to the drawings, a grossing station 10 that is used in autopsy and necropsy procedures is shown according to the present invention. It should be noted that the grossing station 10 may be used on any known autopsy, necropsy, trimming, pathology, or any other procedure performed on tissues or the like, all of which are for use in hospitals, morgues, funeral homes, university laboratories, other laboratories and any other structure that may be used for examining human and animal bodies or human and animal body parts.

The grossing station 10 of the present invention may include a base 12, which generally is arranged on the floor or another surface in the laboratory or room in which the grossing station 10 may be used. It should be noted that it is also contemplated to attach the grossing station 10 to a wall so it is elevated off the floor and capable of movement with respect to the floor.

The grossing station 10 also may include a table 14 or work surface area arranged over a top portion of the base 12. The table 14 may have a generally rectangular shape and a flat work surface arranged on the top end thereof. The grossing station 10 may have a variety of technologies 16 arranged thereon including but not limited to a sink, imaging technologies, and other types of grates, panels or grids, which may be arranged in troughs or on surfaces thereof. A faucet 18 may also be arranged on the table 14 of the grossing station 10 or grossing dissection bench.

The grossing station 10 also may include a hood 20 that may be secured to a top surface of the table 14 of the grossing station 10. The hood 20 generally may have two sidewalls 22 and/or partial sidewalls, a rear wall 24 and a top member 26. The top member 26 when viewed from above generally has a rectangular shape. It should be noted that any necessary lighting 28 and associated electronics to control the lighting may be arranged within or on the surfaces of the hood 20 or on the walls of the hood 20. The rear wall 24 of the hood 20 also may have a plurality of shelves arranged thereon for holding supplies, tools, computer equipment, or the like necessary for the pathology procedure being performed on the grossing station 10. It is also contemplated that the rear wall 24 of the hood 20 may have a metal pegboard 30 to allow for hanging of various components or in order to hold objects needed or used during pathology procedures. It is also contemplated that a first and second panel 32 may be arranged on each end of the first and second sidewall 22 of the hood 20 of the grossing station 10, wherein these panels 32 generally are of a pegboard configuration, made out of metal, which may allow for the placement of shelves in any desired location and/or lights or any other type of material or component needed during the dissection of the material being worked on. It should be note that many other tools and components may be arranged on or near the grossing station 10 of the present invention. The above components are some of those that may be used on the grossing station 10 and the connections and components necessary for the grossing station 10 may be specific to the laboratory needs of the user of the grossing station 10 bearing the technology platform application arranged thereon.

The grossing station software platform 40 of the present invention includes a hardware platform 42 arranged and integrated onto any surface of the grossing station 10. In one contemplated embodiment the hardware platform 42 may comprise a mini computer or dedicated computer 42 that is embedded or integrated within the grossing station 10 or grossing dissection bench that acts as the host for the operating system of the grossing station software platform 40. It should be noted that the hardware platform 42 may also be in the form of a phone or tablet that is wirelessly connected or wired into the grossing station 10 on any surface thereof. This separate mini computer or dedicated computer 42 may be arranged on or near and integrated into the grossing station 10, may enable the installation and validation of the grossing station 10 to be separately validated from any future medical device technology applications that may be added in the future. Many advantages may be gained in validating the connection to the electronic health records separately on the workbench software platform 40 versus having to validate every individual technology running on the workbench at the time of installation. It should be noted that the computer 42 may be connected via any known bracket or any other known system to any surface of the grossing station 10 including but not limited to the hood, walls of the hood, or the top table surface of the grossing station 10. However, it is also contemplated that the mini computer or computer 42 may only be wirelessly connected to the grossing station 10 such that the mini computer 42 may be handheld and placed on a top surface or shelf of the grossing station 10 when not being used by the user of the grossing station 10. The grossing station software platform 40 also may include a common operating system. This common operating system 44 may have a software layer that allows the execution of various application programs tailored for pathology and imaging technologies, and/or any other technologies developed in the future. This operating system 44 is agnostic, meaning it may support a variety of software applications regardless of the specific technology to which they apply. The operating system 44 that may be running on the grossing station 10 and may allow individual applications that lead to new technologies being used on the grossing station 10 to be loaded individually and therefore separately validated into the laboratory process. This may allow for value to be gained by having a host environment runring on the grossing station 10 much like the personal electronics utilized for a smartphone or tablet. Furthermore, any new software technology platform and application may then be loaded and separately validated for use on the software platform 40 of the grossing station 10 making it possible to grow the installed value of the grossing station 10 or workbench over time and not have it linked and tied to a large workflow validation at the time of initial installation of the grossing station 10. This is increasingly advantageous when specific applications of technology are classified as a medical device. These individual medical device applications may then be separately validated as medical device approvals become available and are newly required or approved by the FDA. The grossing station software platform 40 also includes the necessary software application support to allow for future growth of emerging technologies that may not even be on the radar of grossing station users at the present time. The software platform system 40 of the present invention may be capable of loading and supporting individual software applications, which may be separately validated and certified for use according to FDA guidelines or any other required guidelines in the future. The ability for software application support to be available for the grossing station 10 of the present invention may allow the grossing station 10 to have the ability to utilize dissection tools and technologies that exist separately and are not well integrated into the workflow and the hospital network systems, including systems that report directly to the patient record or the electronic health record.

The functionality of the grossing station software platform 40 may allow individual technology applications, such as digital pathology software or imaging tools, to be loaded and validated independently. This structure of having technologies individually loaded and validated may allow for the modular integration of new technology applications to be added to the platform 40 without the need to revalidate the entire grossing station system. Each new application may be validated separately, simplifying the process of regulatory approval needed for such systems in the health care field. Furthermore, the grossing station software platform 40 of the present invention may have EHR integration, wherein the technologies, such as but not limited to, digital imaging cameras or other imaging devices may be easily integrated with hospital EHR systems, thus allowing for seamless documentation updating for patient records in real time. Also, the grossing station software platform 40 may have medical device validation for technologies classified as medical devices. The software platform 40 of the present invention may then provide a way for those applications to be validated individually as per FDA requirements without needing a full workflow validation for the entire system to be performed each time a new technology is added to the grossing station 10. Also, the grossing station software platform 40 of the present invention may future proof the grossing station 10 as to any new technologies and applications that become available. This future proofing of the software platform may allow for easy upgrades and additions including software updates without the need for major system overhauls of the grossing station system software or platform 40.

It should be noted that the grossing station 10 including the technology software platform application 40 of the present invention may be used on any technology that is able to be integrated into the software platform 40. Many recent technologies, such as digital cameras may be integrated into the grossing station 10 for use in digital pathology workflows and documentation. These integrated digital cameras may capture high resolution images of tissue for pathology analysis or the like. Many of these digital imaging cameras typically uses software applications capturing the image and then ultimately loaded into the electronic health record. The software application of the present invention may require listing as a medical device by the FDA. Such applications needed to run the digital cameras that are integrated into the grossing station 10 may be needed to be individually validated into the laboratory workflow depending on the design of the digital camera. It should also be noted that future technology applications may also be integrated into the grossing station software platform 40. Such technologies 16 may be those that enable alternative forms of imaging of the tissue sample. These may include shortwave infrared imaging cameras (SWIR) for tissue analysis, microCT cameras, which are micro computed tomography systems for high resolution internal image of the sample being dissected. The grossing station software platform 40 may also include ultrasound probes, which may be used for tissue imaging and analysis. The new and emerging technologies may also include x-ray imaging systems, which are used for non-evasive imaging of the tissues or samples. The new technologies may also include a biofluorescent or bioluminescence camera, which may be used for imaging specific biomarkers and the tissues samples being dissected. It should be noted that each of these technologies require its own application, which may be loaded and validated independently in the grossing station technology software platform 40 via the application that runs with that software platform. Each new technology added to the grossing station 10 has its own software application 50 that needs validation into the workflow, the grossing station software platform 40 would streamline the separate technology buildout as the grossing station 10 capabilities grow in the future and as medical technology grows.

The grossing station software platform 40 facilitates the integration of various emerging technologies into the grossing station 10 by allowing for modular, individual validation of each technology, the platform 40 simplifies the regulatory process and ensures that the grossing station 10 may be continually updated as new tools and technologies become available. This is a significant advantage in the medical field, particularly for pathology laboratories where regulatory compliance and technological advancements are critical.

The present invention has been described in an illustrative manner. It is to be understood that the terminology, which has been used as intended to be in the nature of words of description rather than that of limitation.

Many modifications and variations of the present invention are possible in light of the above teachings. Therefore, within the scope of the appended claims, the present invention may be practiced otherwise than as specifically described.

What is claimed is:

1. A grossing station software platform for use in integrating workflow technologies into an electronic health record (EHR), comprising:
a host operating system that allows for the independent loading, installation and validation of individual technology applications on a single grossing station.

2. The software platform of claim 1, wherein said host operating system is agnostic, permitting the addition of new technology applications without requiring the revalidation of the entire single grossing station.

3. The software platform of claim 2, wherein each said technology application may be separately validated in accordance with FDA medical device approval requirements.

4. The software platform of claim 1, wherein the grossing station supports integration of various emerging technologies including digital cameras for digital pathology.

5. The software platform of claim 1, wherein the grossing station supports integration of various emerging technologies including shortwave infrared cameras.

6. The software platform of claim 1, wherein the grossing station supports integration of various emerging technologies including advanced imaging microCT systems.

7. The software platform of claim 1, wherein the grossing station supports integration of various emerging technologies including ultrasound probes.

8. The software platform of claim 1, wherein the grossing station supports integration of various emerging technologies including x-ray imaging systems.

9. The software platform of claim 1, wherein the grossing station supports integration of various emerging technologies including biofluorescent or bioluminescent cameras.

10. The software platform of claim 1, wherein future releases of previously validated technology applications may be uploaded to the host operating system as they become available.

11. The software platform of claim 1 further compromising a separate computer arranged on or near said single grossing station, wherein said separate computer is integrated into said single grossing station.

12. The software platform of claim 1 further comprising a mini computer arranged on or near said single grossing station, wherein said mini computer is integrated into said single grossing station.

13. The software platform of claim 1, wherein said host operating system is unique to said single grossing station.

14. A method of integrating technology applications into a grossing station, comprising the steps of:
installing a host operating system on the grossing station;
loading individual technology applications onto a software platform of the grossing station; and
separately validating each of the technology applications in compliance with relevant regulatory guidelines.

15. The method of claim 14 further comprising the step of integrating a separate computer or mini computer into the grossing station.

16. The method of claim 14, wherein said loading step occurs as new technology is added to the grossing station without also having to validate all existing technology on the grossing station.

17. The method of claim 14, wherein said loading step allows new technology to be validated and connected to an electronic health record separately via the software platform.

18. The method of claim 14 further comprising the step of uploading future releases of previously validated applications as they become available to the grossing station.

19. The method of claim 14 further comprising the step of integrating workflow technologies into an electronic health record.

20. The method of claim 19, wherein said technologies enable alternative forms of imaging tissue.
